(19) United States
(12) Patent Application Publication
(10) Pub. No.: US 2012/0071563 A1
(43) Pub. Date: Mar. 22, 2012

(54) STABILIZER

(75) Inventor: Christian Köpsel, Weinheim (DE)

(73) Assignee: BASF SE, Ludwigshafen (DE)

(21) Appl. No.: 13/198,909

(22) Filed: Aug. 5, 2011

Related U.S. Application Data
(60) Provisional application No. 61/370,816, filed on Aug. 5, 2010.

(51) Int. Cl. A61K 31/015; A23D 9/00; A23L 3/349; A23L 1/303; C08B 31/00; A61K 8/73

(52) U.S. Cl. 514/763; 536/102; 426/654; 426/73; 426/601; 426/321; 426/648

(57) ABSTRACT

The present invention relates to a stabilizer comprising starch that comprises at least one ester group corresponding to the formula (I)

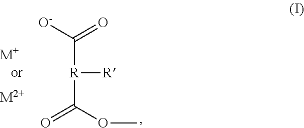

wherein
M⁺ is an alkali metal, M²⁺ is an alkaline earth metal,
R is an alkylene radical and
R′ is an alkyl or alkenyl group having 5 to 18 carbon atoms and less than 0.1% by weight of free alkyldicarboxylic acid compounds or alkenyldicarboxylic acid compounds or mixtures thereof, based on the total weight of the starch,
a method for the production thereof, and also to the use thereof for increasing the stability of a substance that is to be stabilized.

### CROSS-REFERENCE TO RELATED APPLICATIONS

This application claims benefit (under 35 USC 119(e)) of U.S. Provisional Application 61/370,816, filed Aug. 5, 2010 which is incorporated by reference.

### BACKGROUND OF THE INVENTION

The present invention relates to a stabilizer comprising starch that comprises at least one ester group corresponding to the formula (I)

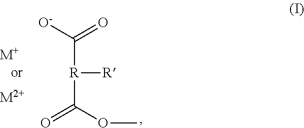

wherein

M⁺ is an alkali metal, M²⁺ is an alkaline earth metal,

R is an alkylene radical and

R′ is an alkyl or alkenyl group having 5 to 18 carbon atoms and

less than 0.1% by weight of free alkyldicarboxylic acid compounds or alkenyldicarboxylic acid compounds or mixtures thereof, based on the total weight of the starch,

a method for production thereof, and also to use thereof for increasing the stability of a substance that is to be stabilized.

In addition, the present invention relates to the use of at least one thus stabilized substance in animal feeds, foods, food supplements, cosmetics or pharmaceuticals. Furthermore, the invention relates to substances that comprise a composition comprising the stabilizer according to the invention and a substance that is to be stabilized.

n-Octenylsuccinate starch is used as an emulsifier in a number of oil-in-water emulsions in the food sector, in the pharmaceutical industry and in the industrial sector (Trubiano, 1986:

Succinate and substituted succinate derivates of starch. In O. B. Wurzburg (Editor) Modified starches: Properties and uses, pages 131-147. CRCPress. Inc. Boca Raton, Fla., US).

An n-octenylsuccinate starch which is permitted for the food sector is one having a maximum degree of substitution with n-octenylsuccinic acid of 3% and free, noncovalently bound, n-octenylsuccinic acid of less than 0.3% (doctoral thesis by Yanjie Bai, Kansas State University, 2008).

WO2007/090610 describes the use of n-octenylsuccinate starch in a composition that contains β-carotene.

WO2007/090614 describes an n-octenylsuccinate starch in which the substances having a molecular weight between 150 Da and 500 kDa are separated off, and the use thereof as an additive in animal and human nutrition and also in cosmetics and pharmaceuticals.

Drusch et al. (J. Agric. Food Chem. 2007, 55, 11044-11051) describe a study for determining the oxidative stability of microencapsulated fish oil. The microcapsules which contain, inter alia, n-octenylsuccinate starch, were exposed to hydroperoxide and propanal.

### A SUMMARY OF THE INVENTION

The invention relates to a stabilizer comprising starch which comprises at least one ester group of the formula (I)

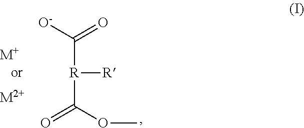

wherein
M⁺ is an alkali metal, M²⁺ is an alkaline earth metal,

R is an alkylene radical and
R′ is an alkyl or alkenyl group having 5 to 18 carbon atoms and the stabilizer comprises less than 0.1% by weight of free alkyldicarboxylic acid compounds or alkenyldicarboxylic acid compounds or mixtures thereof, based on the total weight of the starch.

### A BRIEF DESCRIPTION OF THE FIGURES

FIG. 1 illustrates crevice corrosion after storage of the composition 3 in an aluminum package.

FIG. 2 illustrates pitting after storage of the composition 3 in an aluminum package.

### A DETAILED DESCRIPTION OF THE INVENTION

The object of the present invention was to provide a novel starch-based stabilizer. This should be suitable, in particular, for products which are required in animal or human nutrition, or are used in the field of cosmetics and pharmaceuticals.

The focus of attention was particularly a stabilizer that should be able to increase the stability of diverse substances that are to be stabilized.

In addition, the stabilizer should be suitable for rendering a substance that is to be stabilized inert toward other materials. In particular, storage of a substance thus stabilized in aluminum packages should be made possible.

Accordingly, the stabilizer described at the outset has been found.

The stabilizer according to the invention can increase the stability of a substance that is to be stabilized, e.g. a solid or a liquid. Preferably, “stability” in the context of the present invention is taken to mean the chemical stability of a liquid substance that is to be stabilized. The stability of one or more parameters can be increased in this case. For example, this can be the color characteristics or the particle size and/or the distribution thereof. This also comprises the inertizing action which the stabilizer according to the invention can have on a substance that is to be stabilized. This means that the thus stabilized substance does not react with other materials, or reacts only to a small extent. These can be, for example, metals. Preferably, it is rendered inert toward aluminum or iron or the corresponding alloys or mixtures thereof, particularly preferably toward aluminum or aluminum alloys as are present, for example, in an aluminum package.

The stabilizer according to the invention comprises starch, as described above. The starch can also be a mixture of various such starches.

A starch suitable according to the invention is primarily a starch based on a polysaccharide having the formula (C₆H₁₂O₅)ₙ. It generally comprises D-glucose units which are linked to one another via glycosidic bonds. The macromolecular complex can comprise two components: amylose and amylopectin. Amylose is a linear chain having helical (screw-type) structure which are only linked α-1,4-glycosidically. Amylopectin has an intensely branched structure having α-1,6-glycosidic bonds and α-1,4-glycosidic bonds. The content of amylopectin and amylose in the starch varies and depends on the botanical source from which the starch is isolated.

The starch can be isolated from any possible botanical source such as from wheat, corn, potatoes, cereals, sago, rice or tapioca. However, the starch can also be a mixture of starches from various botanical sources. Preferably these are tapioca starch or waxy corn starch or a mixture thereof. Very particular preference is given to waxy starch.

The starch can also be isolated from genetically modified botanical sources. Likewise, the starch can originate from a mixture of genetically modified sources and non-genetically modified sources.

The stabilizer according to the invention comprises starch which comprises at least one ester group corresponding to the formula (I)

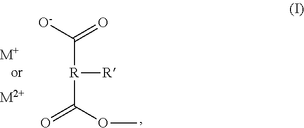

wherein M⁺ is an alkali metal, M²⁺ is an alkaline earth metal, R is an alkylene radical and R′ is an alkyl or alkenyl group having 5 to 18 carbon atoms.

If more than one ester group is present, these can be identical or of different structure. Preferably, the starch comprises more than one ester group. These preferably each have the same structure.

As counterions M⁺, those which come into consideration are, especially, counterions of alkali metals which are derived from lithium, sodium or potassium or, if more than one ester group is present, a mixture thereof.

Preferred counterions M²⁺ are derived from magnesium or calcium or, if more than one ester group is present, a mixture thereof.

When more than one ester group is present, it is also possible that a mixture of alkali metal ions and alkaline earth metal ions are present as counterions. Preferably, the counterion or counterions is/are either M⁺ or M²⁺. A particularly preferred counterion is Na⁺.

Preferred alkylene radicals R have only R′ as side chain. Generally, the chain length of the alkylene radicals can vary within wide limits. Usually it is from 1 to 8 carbon atoms. Preferably, it is in the range from 1 to 5 carbon atoms, particularly preferably from 2 to 4, more particularly preferably two carbon atoms.

Not only the alkyl group but also the alkenyl group R′ can be branched or unbranched, preferably it is unbranched in each case. The chain length of the alkyl group or alkenyl group is preferably 5 to 10 carbon atoms, preferably 6 to 8 carbon atoms, very preferably 8 carbon atoms.

The degree of substitution of the starch suitable according to the invention, that is the number of esterified hydroxyl groups in comparison with the number of free non-esterified hydroxyl groups, varies in the range from 0.1 to 3%, based on the total number of hydroxyl groups present in the starch. The starch suitable according to the invention can also be a mixture of different degrees of substitution.

In a preferred embodiment, the starch suitable according to the invention is an n-octenyl-succinate starch. For producing the octenylsuccinate starch, starch can be treated with cyclic n-octenylsuccinic anhydride. A production method is described, inter alia, in U.S. Pat. No. 2,661,349.

In a very particularly preferred embodiment, the starch suitable according to the invention is a sodium n-octenylsuccinate starch.

Octenylsuccinate starches which are currently commercially available are, for example, Capsul® (sodium octenylsuccinate starch) from National Starch, Cleargum CO 01 from Roquette or Purity® Gum 2000 (sodium octenylsuccinate starch) from National Starch, in particular a sodium octenylsuccinate starch such as Purity® Gum 2000.

The starch that is suitable according to the invention can have further modifications apart from the esterification. The further modifications can be made before or after esterification. The further modifications can be made via chemical reactions, enzymatic or physical modifications, or a combination of these methods.

The starch can be, for example, in part (partially) enzymatically hydrolyzed. The enzymatic partial hydrolysis can proceed via known methods using enzymes, for example using glycosylases and/or hydroxylases. In addition to the enzymatic partial hydrolysis, chemical partial hydrolysis can also proceed. Methods for this purpose are known to those skilled in the art.

The stabilizer according to the invention comprises less than 0.1% by weight of free alkyl-dicarboxylic acid compounds or alkenyldicarboxylic acid compounds or mixtures thereof, based on the total weight of the starch present in the stabilizer.

Alkyldicarboxylic acid compounds or alkenyldicarboxylic acid compounds are preferably taken to mean alkyldicarboxylic or alkenyldicarboxylic acids, salts thereof and anhydrides thereof. These can be present either in dissociated or else in nondissociated form.

The feature “free”, in the context of the present invention, is taken to mean non-covalently bonded.

In a preferred embodiment, the stabilizer according to the invention comprises less than 0.1% by weight of free n-octenylsuccinic acid compounds, in particular n-octenylsuccinic acid, n-octenylsuccinic acid sodium salt, or n-octenylsuccinic anhydride, based on the total weight of the starch present in the stabilizer.

In a further preferred embodiment, the stabilizer according to the invention does not comprise any free alkyldicarboxylic acid compounds or alkenyldicarboxylic acid compounds.

The feature of “not” comprising, in the context of the present invention, is taken to mean that the stabilizer according to the invention comprises alkyldicarboxylic acid compounds or alkenyldicarboxylic acid compounds or mixtures thereof below the limit of detection. The detection of alkyldicarboxylic acid compounds or alkenyldicarboxylic acid compounds is described in the experimental part of the present application. This detection proceeds via a methanol extraction of the starch and subsequent determination of the content of free alkyldicarboxylic acid compounds or alkenyldicarboxylic acid compounds or mixtures thereof by means of liquid chromatography.

In a particularly preferred embodiment, the stabilizer according to the invention does not comprise any free n-octenylsuccinic acid compounds.

The stabilizer, in addition to the starch present according to the invention, can comprise one or more further stabilizing compounds. These stabilizing compounds are preferably compounds which, according to use of the stabilizer according to the invention, are permitted for the corresponding field of use. Corresponding to their permission, these stabilizing compounds are subdivided into various categories.

The stabilizing compounds can be fat soluble or water soluble.

These stabilizing compounds can be compounds for oxidative stability, such as ascorbic acid, esters thereof or salts thereof, water-soluble polyphenols such as, for example, hydroxytyrosol and oleuropein, aglycone, epigallocatechingallate (EGCG), t-butylhydroxytoluene, t-butylhydroxyanisole, citric acid, ethoxyquin, propyl gallate, tertiary butylhydroxyquinoline or 6-ethoxy-1,2-dihydroxy-2,2,4-trimethylquinoline (EMQ) or mixtures thereof.

Furthermore, the stabilizer according to the invention can also comprise stabilizing compounds for microbial stability, for example methyl 4-hydroxybenzoate, propyl 4-hydroxybenzoate, sorbic acid or benzoic acid or salts thereof or mixtures thereof.

In addition, the stabilizer according to the invention can also comprise emulsifiers. Examples of emulsifiers are ascorbyl palmitate, polyglycerol esters of fatty acids such as polyglycerol-3-polyricinoleate (PGPR 90), sorbitan esters of fatty acids, such as sorbitan monostearate (span 60), PEG(20) sorbitol monooleate, propylene glycol esters of fatty acids or phospholipids, such as lecithin, or mixtures thereof.

Stabilizing compounds which can act as a protective colloid can be present in the stabilizer according to the invention. For this purpose, for example the following gelatins come into consideration: beef, pork or fish gelatin, in particular acid- or basic-degraded gelatin having bloom numbers in the range from 0 to 250, very particularly preferably gelatin A 100, A 200, A 240, B 100 and B 200, and also low-molecular-weight enzymatically degraded gelatin types having the bloom number 0 and molecular weights of 15 000 to 25 000 Dal such as, for example, Collagel® A and Gelitasol® P (Stoess, Eberbach) and also mixtures of these gelatin types.

In addition, dextrin, pectin, gum arabic, ligninsulfonates, chitosan, polystyrene sulfonate, alginates, casein, caseinate, methylcellulose, carboxymethylcellulose, hydroxypropylcellulose or mixtures thereof can be used as protective colloid.

The protective colloids can also include plant proteins such as soybean, rice and/or wheat proteins, wherein these plant proteins can be present in partially degraded or non-degraded form.

In the stabilizer according to the invention, the weight ratio of the starch suitable according to the invention to at least one further stabilizing compound is 10:1 to 1:10, preferably 4:1 to 1:4, in particular 3:1 to 1:1.

The stabilizer according to the invention preferably comprises, in addition to the starch present according to the invention, native starch, modified starch, dextrin, pectin, gum arabic, ligninsulfonates, chitosan, polystyrenesulfonate, alginates, casein, caseinate, methylcellulose, carboxymethylcellulose, hydroxypropylcellulose, plant proteins such as soybean, rice and/or wheat proteins, wherein these plant proteins can be present in partially degraded or non-degraded form.

Modified starches in this case are starches having chemical, enzymatic and/or physical modifications which do not comprise an ester group corresponding to the starch present according to the invention. The starches in this case can have one or more modifications.

The invention further relates to a method for producing the stabilizer according to the invention. The production method comprises separating off free alkyldicarboxylic acid compounds or alkenyldicarboxylic acid compounds or mixtures thereof from starch, which comprises at least one ester group of the formula (I)

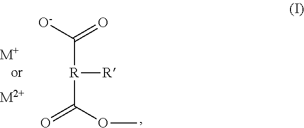

wherein

M⁺ is an alkali metal, M²⁺ is an alkaline earth metal,

R is an alkylene radical and

R′ is an alkyl or alkenyl group having 5 to 18 carbon atoms

by means of reprecipitation down to a residual content of less than 0.1% by weight, based on the total weight of the starch and optionally the addition of one or more further stabilizing compounds. For a more detailed description of the starch and the further stabilizing compounds, reference is made to the preceding text.

The reprecipitation generally proceeds using a medium in which either the starch suitable according to the invention or the free alkyldicarboxylic acid compounds or alkenyldicarboxylic acid compounds are not soluble.

Preferably, the reprecipitation proceeds using a medium in which the starch contained in the stabilizer is not soluble but the free alkyldicarboxylic acid compounds or alkenyldicarboxylic acid compounds do dissolve. In a preferred embodiment, the reprecipitation proceeds using at least one alcohol or a mixture of at least one alcohol and water. In a particularly preferred embodiment, the reprecipitation proceeds from water using ethanol.

In a further preferred embodiment, the free alkyldicarboxylic acid compounds or alkenyl-dicarboxylic acid compounds or mixtures are separated off below their detection limit, i.e. after separation no residual content is present any longer in the stabilizer according to the invention.

For reprecipitating starch using alcohol, in principle methods are available to those skilled in the art which they can apply at present.

The invention further relates to a composition which comprises the stabilizer according to the invention and a substance that is to be stabilized.

A “substance that is to be stabilized” can be a single substance or a mixture of substances that are to be stabilized.

In a preferred embodiment of the invention, the substance that is to be stabilized comprises a fat-soluble substance or a mixture thereof.

The feature “fat soluble”, in the context of the present invention, is taken to mean lipophilic (fat loving). Fat-soluble substances may readily be dissolved in fats and oils.

In a preferred embodiment of the invention, the fat-soluble substance is a carotenoid, a fat-soluble vitamin and/or a PUFA (polyunsaturated fatty acid).

Carotenoids are generally taken to mean the known representatives accessible from natural or synthetic sources. Examples thereof are alpha-carotene, beta-carotene, gamma-carotene, lycopene, lutein, astaxanthin, zeaxanthin, cryptoxanthin, citranaxanthin, canthaxanthin, echinenone, bixin, β-apo-4-carotenal, β-apo-8-carotenal, β-apo-4-carotenoic acid esters, singly or as a mixture. Preferably, a carotenoid is selected from the group consisting of alpha-carotene, beta-carotene, gamma-carotene, astaxanthin, canthaxanthin, citranaxanthin, lycopene and lutein. Very particular preference is given to beta-carotene.

The fat-soluble vitamins generally include vitamins A, E, D and K including derivatives thereof, for example vitamin A esters such as vitamin A acetate, vitamin A propionate or vitamin A palmitate, and also vitamin E esters such as tocopheryl acetate. They can be used in the context of the invention in the form of vitamin solutions in oils, as provitamins and also as pure vitamins of natural or synthetic origin. Preference is given to vitamin A and E and derivatives thereof. Particular preference is given to vitamin A acetate, vitamin A propionate and vitamin A palmitate and vitamin E acetate, and also mixtures thereof, very particular preference is given to vitamin A acetate and/or vitamin E acetate.

The PUFAs (polyunsaturated fatty acids) include polyunsaturated fatty acids, preferably having a chain length of 18, 20 or 22 carbon atoms having 2 to 6 double bonds in the cis conformation. Those of biological relevance are the omega-3 fatty acids and omega-6 fatty acids. In the case of omega-3 fatty acids, the last double bond in the polyunsaturated carbon chain is in the third last—seen from the carboxyl end—C—C bond. The omega-3 fatty acids include alpha-linolenic acid (ALA, 18:3ω-3), eicosapentaenoic acid (EPA, 20:5ω-3) and docosahexaenoic acid (DHA, 22:6ω-3). In omega-6 fatty acids, the last double bond in the polyunsaturated carbon chain is in the sixth last—seen from the carboxyl end—C—C bond. Examples of omega-6 fatty acids are linoleic acid (18:2ω-6, 9Z, 12Z-octadecadienoic acid), gamma-linoleic acid (18:3ω-6, 6Z, 9Z, 12Z-octadecadtrienoic acid), and arachidonic acid (20:4ω-6, 5Z, 8Z, 11Z, 14Z-eicosatetraenoic acid). Preferably, the long-chain polyunsaturated fatty acids are alpha-linolenic acid, eicosapentaenoic acid, docosahexaenoic acid, linoleic acid, gamma-linolenic acid, arachidonic acid or a mixture thereof. Particularly preferably, the long-chain polyunsaturated fatty acids are eicosapentaenoic acid, docosahexaenoic acid or a mixture thereof.

The composition according to the invention can in addition comprise auxiliaries, such as thickeners, hard fats, chelating agents, for example alkali metal or alkaline earth metal salts of citric acid, phytic acid or phosphoric acid and/or emulsifiers, where these are not already present in the stabilizer according to the invention. Examples of emulsifiers are ascorbyl palmitate, polyglycerol esters of fatty acids, such as polyglycerol-3-polyricinoleate (PGPR 90), sorbitan esters of fatty acids, such as sorbitan monostearate (span 60), PEG(20)-sorbitol monooleate, propylene glycol esters of fatty acids, or phospholipids, such as lecithin.

Preferably, the composition according to the invention comprises 5 to 99% by weight of the stabilizer according to the invention and from 5 to 95% by weight of the substance that is to be stabilized, particularly preferably from 40 to 99% by weight of the stabilizer according to the invention and from 1 to 60% by weight of the substance that is to be stabilized, in particular a fat-soluble substance, in each case based on the total weight of the composition.

The composition according to the invention can in addition comprise auxiliaries such as thickeners, hard fats, chelating agents and/or emulsifiers. Preferably, the composition then comprises from 5 to 80% by weight of the stabilizer according to the invention and 1-60% by weight of the substance that is to be stabilized, particularly preferably from 30 to 60% of the stabilizer according to the invention and from 5 to 50% by weight of the substance that is to be stabilized, in particular a fat-soluble substance, in each case based on the total weight of the composition, wherein the sum of the components of the composition yields 100% by weight.

In a further preferred embodiment, the composition according to the invention comprises from 1 to 20% by weight of carotenoid and from 5 to 99% by weight of the stabilizer according to the invention, particularly preferably from 1 to 20% by weight of carotenoid and from 30 to 60% by weight of the stabilizer according to the invention, in each case based on the total weight of the composition. The sum of the components of the composition yields in each case 100% by weight, wherein the composition according to the invention can in addition comprise auxiliaries and/or a further substance that is to be stabilized.

In a further preferred embodiment, the composition according to the invention comprises from 30 to 50% by weight of vitamin A and from 5 to 70% by weight of the stabilizer according to the invention, in particular, in each case based on the total weight of the composition. The sum of the components of the composition yields in each case 100% by weight, wherein the composition according to the invention can comprise in addition auxiliaries and/or a further substance that is to be stabilized, in particular vitamin E.

In a further preferred embodiment, the composition according to the invention comprises from 10 to 60% by weight of vitamin E and from 5 to 90% by weight of the stabilizer according to the invention, particularly preferably from 10 to 60% by weight of vitamin E and from 30 to 60% by weight of the stabilizer according to the invention in each case based on the total weight of the composition. The sum of the components of the composition yields in each case 100% by weight, wherein the composition according to the invention can additionally comprise auxiliaries and/or a further substance that is to be stabilized.

In a further preferred embodiment, the composition according to the invention comprises from 1 to 40% by weight of PUFA and from 5 to 99% by weight of the stabilizer according to the invention, particularly preferably from 1 to 40% by weight of PUFA and from 30 to 60% by weight of the stabilizer according to the invention, in each case based on the total weight of the composition. The sum of the components of the composition yields in each case 100% by weight, wherein the composition according to the invention can additionally comprise auxiliaries and/or a further substance that is to be stabilized.

The invention further relates to a method for stabilizing a substance that is to be stabilized which comprises mixing a stabilizer according to the invention with a substance that is to be stabilized.

The respective mixing method depends on the components that are to be mixed. Those skilled in the art in this case choose a suitable mixing method. The preferred mixing methods include emulsification.

Mixing can proceed, for example, by mixing chamber micronization, emulsification, grinding, extrusion or a combination thereof.

Mixing chamber micronization can proceed, for example, according to the method described in EP 0 065 193. Finely divided pulverulent carotenoid preparations can be produced in that a carotenoid is dissolved in a volatile, water-miscible organic solvent and at elevated temperatures, optionally at elevated pressure, the carotenoid is precipitated by mixing the solution with an aqueous solution of the stabilizer according to the invention, and then drying it.

An emulsification method is described, for example, in DE 12 11 911 or EP 0 410 236, wherein the stabilizer according to the invention can be present in the aqueous solution.

WO 2007/003543 describes, for example, a grinding method in which β-carotene is comminuted as a suspension by grinding in the presence of sucrose or glucose and modified starch to a particle size of about 0.6 μm and the carotenoid-comprising suspension is then converted into a dry powder. The grinding method described can also proceed in the presence of the stabilizer according to the invention.

Extrusion methods are known, for example, from WO 00/21504 and WO 04/009054. In the emulsification method, for example, first an emulsion is formed comprising the substance that is to be stabilized and the stabilizer according to the invention, which emulsion is then extruded with a matrix.

The invention further relates to the use of the stabilizer according to the invention for increasing the stability of a substance that is to be stabilized. For instance the compositions according to the invention can safely come into contact with aluminum- and/or iron-comprising materials.

Using the stabilizer according to the invention makes possible, for example, storage in an aluminum package of a substance that is to be stabilized, since neither pitting nor crevice corrosion occurs. Storage in aluminum packages is of great advantage, compared with, for example plastic packages, especially in the case of light-sensitive and/or oxidation-sensitive substances that are to be stabilized. The storage can extend in this case up to one year. However, it is also possible to store the substance that is to be stabilized or the composition according to the invention for more than one year in an aluminum-comprising and/or iron-comprising package, e.g. up to two years. The storage time can also be longer, however, for example up to three years.

In addition, the clearing (creaming, sedimentation) of the composition according to the invention is improved by using the stabilizer according to the invention. Likewise, owing to the use of the stabilizer according to the invention, no growth of particles occurs during storage, if the composition is a dispersion. The tendency to agglomeration is decreased, or agglomeration does not take place.

In a further preferred embodiment, the substance that is to be stabilized is an additive to an animal feed, food or food supplement and also cosmetic or pharmaceutical.

The composition is suitable, inter alia, as additive to an animal feed or to food preparations or mixed feed, as an agent for producing a pharmaceutical and cosmetic preparation and also for producing a food supplement preparation in the human or animal sector. Preferably, the composition may be used as feed additive in animal nutrition, for example by mixing it into feed pellets during extrusion or by applying or spraying it to feed pellets. The application as feed additive proceeds, in particular, by direct spraying of the composition according to the invention, for example as what is termed “post-pelleting application”. Preferably, the feed pellets are loaded with the compositions under reduced pressure.

Typical fields of use in the food sector are, for example, vitaminization and coloring of beverages, milk products such as yogurt, milkshakes or ice milk, and also blancmange powders, egg products, baking mixtures and confectionery products. In the cosmetics sector, the composition can be used, for example, for decorative body care agents, for example in the form of a cream, a lotion, as a lipstick or makeup.

The invention further relates to a substance which comprises the composition according to the invention. Preferably, the substance is an animal feed, a food, a food supplement, a cosmetic or a pharmaceutical.

The invention will be described by the following examples which do not limit the invention, however:

### EXAMPLES

### Detection of Free Alkyldicarboxylic or Alkylenedicarboxylic Acids

### Extraction and Preparation of the Sample Solution

Approximately 500 mg of starch were extracted with 15 ml of methanol for 12 h with constant shaking. The extraction mixture was filtered. The starch of the filter was washed with 7 ml of methanol. This was repeated three times. All filtrates are combined. Approximately 80% of the free alkylsuccinic acid compounds or alkylenesuccinic acid compounds were extracted in this manner. To the filtrates was added 1 ml of a 0.16 N methanolic KOH solution. The filtrates were dried at 30° C. in a rapid evaporator. The residue was dissolved in 2 ml of methanol. 0.5 ml of this residue solution was transferred to another vessel. To this 0.5 ml of the residue solution were added 0.5 ml of a derivatizing solution (2.8 g of 2-p-dibromoacetophenone and 0.28 g of 18-crown-6 in 50 ml of CH₃CN). After the vessel was closed, it was treated at 80° C. for 30 min. Thereafter, the solution was cooled to room temperature and used within 24 h.

### Analysis by Means of Liquid Chromatography

Column: Micro-Bondapack C18 (Waters) or an equivalent column

Mobile phase: elution with a gradient from 70% methanol in water to 80% methanol in water in the course of 5 minutes

Flow rate: 1.5 ml/min

Detector: UV at 254 nm, attenuation 0.16 AUFS,

Injection volume: 5 μl

### Preparing the Calibration Curve

A 0.5 M solution of sodium octenylsuccinate was produced (solution A). 0.25 ml of the solution A was transferred to another vessel and diluted with methanol (solution B). Three different calibration standards were produced by adding 0.5 ml, 1 ml and 2 ml of solution B to the 0.25 ml of solution A. To each of these mixtures, 1 ml of a 0.16 N methanolic KOH solution was added. Each solution was dried at 30° C. The supernatant was dissolved in each case in 2 ml of methanol (solutions C1, C2 and C3). 0.5 ml of these residue solutions was transferred to another vessel. To these 0.5 ml samples of the residue solutions were added 0.5 ml of a derivatizing solution (2.8 g of 2-p-dibromoacetophenone and 0.28 g of 18-crown-6 in 50 ml of CH₃CN). After the vessel was closed it was treated at 80° C. for 30 min. Thereafter the solutions were cooled to room temperature. 5 μl were used in each case for the liquid chromatography. The content of residues in this 5 μl was:

for solution C1 0.2375 μg

for solution C2 0.4750 μg

for solution C3 0.9500 μg

### Calculation:

A calibration curve corresponding to the method described above was prepared. On the basis of the height of the peak of the unknown solution in the liquid chromatography, the content of free alkyl and alkylene compounds (calculated as octenylsuccinic acid) was determined in the injection volume using the calibration curve.

% of free alkyl and alkylene compounds: 300*value from the graph/weight of the starch (mg)

Note: The formula was corrected to 100% yield by dividing by 0.8 (240/0.8=300)

### Example 1

Production of composition 1 (according to the invention): composition which does not comprise any free n-octenylsuccinic acid compound

380 g of water were initially charged and heated to 60° C. To this were added 117 g of reprecipitated (does not contain any free alkyldicarboxylic acid compounds or alkenyl-dicarboxylic acid compounds) Purity Gum 2000 (National Starch), 175 g of glucose and 5.6 g of sodium ascorbate. The mixture (solution L4) was allowed to swell at 60° C. for 60 minutes.

18.7 g of β-carotene dispersion (Lucarotin® 20 MCT, BASF) were heated on an oil bath heated to 180° C. until the β-carotene was completely dissolved. The oil phase was then introduced into the water phase with stirring using a toothed wheel rim dispersion machine (Ultra Turrax) at 10 000 rpm. After an emulsification time of 15 minutes, the emulsion was fine-emulsified by means of a high-pressure homogenizer at 700 bar. After spray drying a free-flowing cold-water-dispersible powder was obtained having a β-carotene content of 1.15%.

Production of composition 2 (for comparison): composition having 0.1% by weight of free n-octenylsuccinic acid compounds

The production corresponds to the production of composition 1, wherein additionally, 0.51 g of 23% strength octenylsuccinic acid sodium salt was added to the solution L4.

Production of composition 3: composition having 0.5% by weight of free n-octenylsuccinic acid compounds (comparative example)

The production corresponds to the production of composition 1, wherein 2.55 g of 23% strength octenylsuccinic acid sodium salt was additionally added to solution L4.

Of the compositions 1, 2 and 3, the particle size and the clearing were measured before and after storage at room temperature for 5 h. The clearing was measured by means of a Lumifuge (Dispersion Stability and Particle Characterization by Sedimentation Kinetics in a Centrifuge Field, D. Lerche, Journal of Dispersion Science and Technology Vol. 23, no. 5, pp. 699-709; Rapid assessment of sedimentation stability in dispersions using near infrared transmission measurements during centrifugation and oscillatory rheology M. Kuentz, D. Rothlisberger, European Journal of Pharmaceutics 56 (2003) 355-361). The particle size was measured by light scattering. The results are listed in table 1.

TABLE 1
Results of measurements of particle size and clearing before and
after storage for 5 h at room temperature.

| | Composition 1 | Composition 2 | Composition 3 |
|---|---|---|---|
| | (according to the | (comparative | (comparative |
| | invention) | example) | example) |
| Particle size | 160 | 157 | 161 |
| without storage | | | |
| [nm] | | | |
| Particle size after | 165 | 168 | 177 |
| storage for 5 h at | | | |
| RT [nm] | | | |
| Δ particle size | 5 | 11 | 16 |
| Clearing without | 17.6 | 14.5 | 15.9 |
| storage [%] | | | |
| Clearing after | 21.1 | 21.1 | 33.8 |
| storage for 5 h at | | | |
| RT [%] | | | |
| Δ clearing | 3.5 | 6.6 | 17.9 |

### Example 2

Storage of a composition comprising the stabilizer according to the invention and β-carotene in an aluminum package.

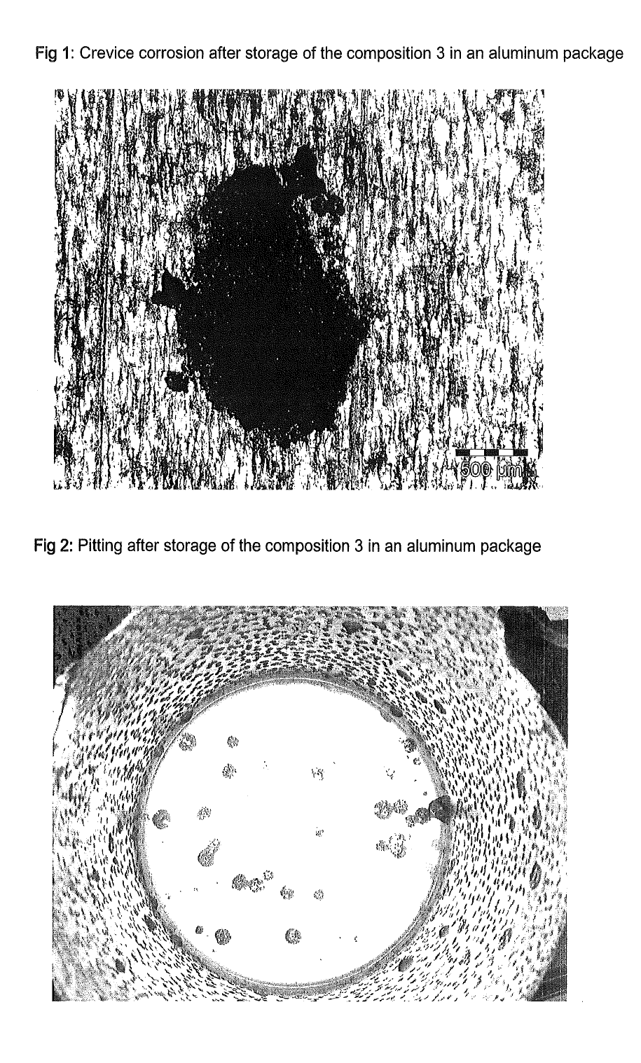

Compositions according to compositions 3 (comparative example) and 1 (example according to the invention) were stored in an aluminum package at room temperature over a period of 14 days. In the comparative example in which no stabilizer according to the invention was used, after storage, a change in the aluminum package was observed. The storage leads to a greater crevice corrosion on the aluminum package (2 to 3 mm; see FIG. 1) and pitting, in particular on the side walls of the aluminum package (see FIG. 2). In the composition in which the stabilizer according to the invention had been used, in contrast, no change of the aluminum package was observed.

1-16. (canceled)

17. A stabilizer comprising starch which comprises at least one ester group of the formula (I) 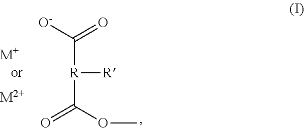
wherein
M⁺ is an alkali metal, M²⁺ is an alkaline earth metal,
R is an alkylene radical and
R′ is an alkyl or alkenyl group having 5 to 18 carbon atoms and
the stabilizer comprises less than 0.1% by weight of free alkyldicarboxylic acid compounds or alkenyldicarboxylic acid compounds or mixtures thereof, based on the total weight of the starch.

18. The stabilizer according to claim 17, wherein the stabilizer does not comprise any free alkyldicarboxylic acid compounds or alkenyldicarboxylic acid compounds.

19. The stabilizer according to claim 17, wherein the starch is a sodium n-octenylsuccinate starch.

20. A method for producing the stabilizer according to claim 17 comprising separating off free alkyldicarboxylic acid compounds or alkenyldicarboxylic acid compounds or mixtures thereof from starch, according to claim 17, by means of reprecipitation down to a residual content of less than 0.1% by weight, based on the total weight of the starch.

21. The method according to claim 20, wherein the precipitation proceeds using ethanol.

22. The method according to claim 20, wherein, after the separation, no residual content of free alkyldicarboxylic acid compounds or alkenyldicarboxylic acid compounds is present in the stabilizer.

23. A composition comprising a stabilizer according to claim 17 and a substance that is to be stabilized.

24. The composition according to claim 23, wherein the substance that is to be stabilized comprises a fat-soluble substance.

25. The composition according to claim 24, wherein the substance that is to be stabilized comprises at least one carotenoid, one fat-soluble vitamin, one PUFA (polyunsaturated fatty acid) or a mixture thereof.

26. The composition according to claim 25, wherein the substance that is to be stabilized comprises astaxanthin, alpha-carotene, beta-carotene, gamma-carotene, vitamin A, vitamin E, PUFA, lutein, lycopene or a mixture thereof.

27. A method for stabilizing a substance that is to be stabilized which comprises mixing at least one stabilizer according to claim 17 with a substance that is to be stabilized.

28. An additive to an animal feed, food, food supplement, cosmetic or pharmaceutical which comprises the stabilizer according to claim 17.

29. The use according to claim 28, wherein the substance that is to be stabilized is an additive to an animal feed, food, food supplement, cosmetic or pharmaceutical which comprises the stabilizer according to claim 17.

30. A process for rendering a substance that is to be stabilized inert toward iron, aluminum or an iron-comprising and/or aluminum-comprising alloy comprises utilizing the stabilizer according to claim 17.

31. A substance comprising a composition according to claim 23.

32. The substance according to claim 31, wherein the substance is an animal feed, a food, a food supplement, a cosmetic or a pharmaceutical.
